(19) United States
(12) Patent Application Publication
(10) Pub. No.: US 2014/0067023 A1
(43) Pub. Date: Mar. 6, 2014

(54) OPTICAL NEURON STIMULATION PROSTHETIC USING SILICON CARBIDE

(71) Applicants: Joseph J. Register, Tampa, FL (US); Stephen E. Saddow, Odessa, FL (US); Christopher L. Frewin, Tampa, FL (US); Edwin Weeber, Apollo Beach, FL (US)

(72) Inventors: Joseph J. Register, Tampa, FL (US); Stephen E. Saddow, Odessa, FL (US); Christopher L. Frewin, Tampa, FL (US); Edwin Weeber, Apollo Beach, FL (US)

(73) Assignee: UNIVERSITY OF SOUTH FLORIDA, Tampa, FL (US)

(21) Appl. No.: 13/953,224

(22) Filed: Jul. 29, 2013

Related U.S. Application Data
(63) Continuation of application No. PCT/US2012/023131, filed on Jan. 30, 2012.
(60) Provisional application No. 61/437,346, filed on Jan. 28, 2011.

(51) Int. Cl. A61N 5/06

(52) U.S. Cl. CPC A61N 5/0622
USPC 607/89; 607/92; 29/428

(57) ABSTRACT

The microfabricated prosthetic device uses local, direct, and wavelength-specific optical stimulation to achieve an action potential from a single or small group of neurons within the central nervous system (CNS). The device is biocompatible, mechanically flexible, and optically transparent. The device can also use integrated electrodes for additional input/output (IO) locations, signal verification, feedback, wireless communication, and characterization of the electrochemically-evoked potential received from the activated neuron. The purpose of the device is to act as a neural interface prosthetic. The prosthetic is designed as the central component of a brain machine interface (BMI).

### CROSS REFERENCE TO RELATED APPLICATIONS

This application is a continuation of prior filed International Application, Serial Number PCT/US2012/023131, filed Jan. 30, 2012, entitled “Optical Neuron Stimulation Prosthetic Using SiC (Silicon Carbide)” which claims priority to U.S. Provisional Patent Application No. 61/437,346 filed Jan. 28, 2011, entitled “Optical Neuron Stimulation Prosthetic Using SiC (Silicon Carbide)”. The content of each of which is incorporated herein by reference.

### FIELD OF THE INVENTION

This invention relates to a device and method for optical neuron stimulation prosthetics using silicon carbide (SiC).

### BACKGROUND OF INVENTION

The importance of controlling action potentials in certain cell types was emphasized first by Francis Crick in his Kuffler Lectures at the University of California in San Diego 2002. Soon after this talk, an explosion of genetic targeting experiments culminated in the Deisseroth group genetically isolating a photosensitive ion channel called Channelrhodopsin-2 (ChR2). The team then used standard genetic engineering techniques to insert the needed DNA sequence into a neuron using an AAV virus. The process resulted in the world's first mammalian neuron with a blue-green light controlled action potential. ChR2 is still the most commonly used modification in optogenetics, but several other ion channels have now been discovered. Most notably, the antithesis to ChR2, Natronomonas pharaonis halorhodopsin (NpHR) is an ion pump for chlorine ions. When this structure is illuminated with yellow light an inhibitory current is created that effectively silences the neuron. Between these two ion-channel structures total bipolar state control over the action potential can be realized. This type of optical targeting and stimulation provides a great advantage over traditional electrical stimulation (See Alexander M Aravanis, Li-Ping Wang, Feng Zhang, Leslie A Meltzer, Murtaza Z Mogri, M Bret Schneider and Karl Deisseroth. “An optical neural interface: in vivo control of rodent motor cortex with integrated fiberoptic and optogenetic technology” J. Neural Eng. 4, S143, 2007). Precise light control by lenses and other optical structures results in high spatial resolution while the genetic targeting technique provides neuron differentiation. Using a localized light emitter, CNS cells can be activated, inhibited, and intercell signaling can be controlled on an individual neuron level with millisecond temporal resolution. Currently, these devices are extremely lifetime limited. Neural prosthetics have to survive the rigors of the body's harsh environment and better materials are warranted if implants are to have design-lifetimes on the order of a human lifetime.

The implantation of a prosthetic device in the brain leads to a “foreign body” response mounted by the immune system. This immunologic response, coupled with the chemically harsh environment of the body, quickly degrades neural prosthetics in-vivo. Beginning from the moment of implantation, performance begins to degrade and eventually terminates in total failure. This process usually happens in a matter of weeks to years. Previously, the effect could only be mitigated temporarily using a careful selection of encapsulation materials. In order to enhance device lifetimes and reduce inflammation designers utilize materials such as sapphire, ceramics, glass, and polymers to fend off device destruction. To complicate the issue, electrically active prosthetics such as wireless and optoelectronic devices use these materials as a protective coating to encapsulate otherwise poisonous electronic components.

The specialized biomaterials used to build neural prosthetics have a dual purpose. First, the materials reduce the amount of immunologic response caused by the prosthesis. Second, these materials provide a barrier between the body and the prosthetic in an attempt to save the body from undue exposure to otherwise toxic substances. In the case of an optical neural prosthesis, the most devastating of these effects is a glial scar that forms around the device effectively encapsulating it in dense fibrous tissue.

The glial scar disrupts the light transmitted from the device causing both optical path loss and degraded spatial resolution. Common biomaterials, such as sapphire, some ceramics, and glass may not dissolve or dissolute in the body but result in a large immunologic response when implanted in neural tissue. Polymers, such as polyimide, work well initially but eventually crack and fail. The instant invention addresses these concerns by using carefully selected polytypes (crystal structures) of single crystalline silicon carbide (SiC) material. It has been shown that 3C-SiC does not crack or swell over time like many polymers and its use can result in a device that is wholly biocompatible and mechanically rigid enough to withstand chronic implantation. In addition 3C-SiC has an exceedingly low immune response in chronic cortical implantation.

Single crystal SiC has been shown to have a very low immune response, high mechanical resilience, and possess optical transparency at certain key wavelengths currently used in optogenetics therapy (Gary Lynn Harris: “Properties of silicon carbide”, INSPEC, Institution of Electrical Engineers, 282 pp, ISBN 0 85296 870 1, pp. 16-17, 1995 and Garret D Stuber “Dissecting the neural circuitry of addiction and psychiatric disease with optogenetics” Neuropsychopharmacology, 35, pp. 341-342, 2010). Using silicon carbide as a primary construction material for the optical stimulation prosthesis allows for device lifetimes that are orders of magnitude greater than current standards.

### SUMMARY OF INVENTION

The present invention provides a microfabricated prosthetic device that uses local, direct, and wavelength-specific optical stimulation to evoke an action potential from a single or small population of neurons within the central nervous system (CNS). The device topology is based off of a preexisting design developed by researchers at the University of Michigan (John Seymour, Mayurachat Ning Gulari, Joerg Lahann, Daryl Kipke “Method for Manufacturing an Implantable Electronic Device”, U.S. Patent Application No. 2010/0145422) and drastically improved by the novel application of SiC biomaterials. The invention is presented in multiple topologies demonstrated as embodiments herein. The purpose of the device is to act as a neural interface prosthetic whereby the prosthetic is a central component of a brain machine interface (BMI) system.

Using the previously established ISO-10993 standard it has been established that 3C-SiC can be used as a highly biocompatible material with little immune response (Christopher L. Frewin, “The neuron-silicon carbide interface: Biocompatibility study and BMI device development” PhD dissertation, University of South Florida, 2009). It has also been established that other polymorphs of SiC such as 4H-SiC and 6H-SiC show low toxicity to immortalized mammalian cells in tests (See C. Coletti, M. J. Jaroszeski, A. Pallaoro, A. M. Hoff, S. Iannotta, and S. E. Saddow, “Biocompatibility and wettability of crystalline SiC and Si surfaces”, IEEE EMBC Proceedings, vol. 22-26, 2007, pp. 5849-5852). Using these polymorphs of silicon carbide in concert can render a device that is functional, non-toxic, and extremely resistant to mechanical failure.

Single crystal SiC has several properties that make it well suited for optical neuron stimulation. It can be used to create integrated electronic circuitry, light emitters, and on-chip optical waveguide structures, and by using doped regions of epitaxial SiC light emitting junctions can be realized. Conductors can be formed on/within the device by using a high temperature process to create epitaxial graphene or through the creation of highly doped n++ or p++ regions. Overall, the material properties of SiC can be harnessed to create a long-term optostimulation and neural recording solution for the invention presented. Therefore, SiC is used not only due to its superior biocompatibility, but also because it can produce the desired light and provide the necessary the heat dissipation.

The first aspect of the device involves the transmission and delivery of light to the targeted neural tissue. One embodiment of the device uses micromachined layers of single crystal SiC polytypes to form long, thin, longitudinal waveguides that carry at least a portion of the light from a source near the support down the longitudinal axis to targeted neural tissue along the length of the device (FIG. 1). The optical waveguides are fabricated with a combination of grown epitaxial films consisting of 6H-SiC, 4H-SiC, 3C-SiC polytypes with the 3C-SiC polytype always used as the outer most device layer to ensure the highest level of biocompatibility (See Christopher L. Frewin, “The neuron-silicon carbide interface: Biocompatibility study and BMI device development” PhD dissertation, University of South Florida, 2009).

In this embodiment either standard LED emitters are fabricated onto the Si support, i.e., not in direct contact with neurons, or outside the dura but still beneath the skull or micro fabricated semiconductor lasers are utilized as emission sources.

Yet another embodiment of this device uses light emitters directly fabricated at points along the length of the prosthetic (FIG. 2). This configuration eliminates the need to guide light down the length of the prosthetic using waveguides but requires conductive traces to power each light emitting diode (LED) structure to run along the length of the device. Commonly, optical stimulation prosthetics with integrated LEDs are made using cytotoxic semiconductors such as GaAs. Often, these materials are coated in biocompatible polymers such as polyimide, but this leads to device failure due to the swelling and cracking that these coating materials undergo in the long-term. This failure causes the eventual release of toxic materials into the body.

Another aspect of the device involves electrical feedback taken directly from the optically activated neural tissue. The electrical neuronal feedback can be sensed by direct electrical conduction of a microelectrode or induced by other means such as micro induction coil or similar device in place of the microelectrode. Each voltage or field effect sensor is connected to a conductive trace providing a feedback path.

In another embodiment, the device employs an array of electrical feedback microelectrodes to detect the presence of electrical neural activation potentials evoked by the light emitters. Each microelectrode is connected to a conductive trace providing a feedback path. It is envisioned that each optical emitter (LED or waveguide) will have at least one microelectrode nearby, typically less than 20 um or less than the average width from the targeted neuron type and each microelectrode will have a conductive return trace to route the electrical signal down the length of the device terminating near the top support structure.

Another aspect of the device is the processing, communication, and drive circuitry that is all located in the device support structure. Here, optical outputs are sent down the length of the prosthetic and electrical feedback from recording electrodes can optionally be recorded or passed on to an external interface.

Many embodiments of the of the device contain general processing and support circuitry that handles the addressing and control of light emitters as well as the processing of feedback emanating from the microelectrodes on the device which we refer to henceforth as an application specific integrated circuit (ASIC).

In some embodiments of the invention, the transmission circuitry can include means such as inductive coupling or a RF subsystem of wireless communication. In addition, the invention may include a receiver allowing bi-directional RF communication. The same system may be used for both transmitting and receiving. It is also envisioned that the same RF subsystem used for communication, can wirelessly transmit power to the prosthetic's drive circuitry powering the unit through inductive coupling. Such circuitry would be kept outside the dura and not in direct contact with neural tissue.

In this same aspect, an embodiment would contain a method of communication by using an external wire (or wires) exiting the prosthetic and subject's body where it is to be connected to further interface systems. With this embodiment long-term power and communication would all be conducted through the wire.

### BRIEF DESCRIPTION OF THE DRAWINGS

For a fuller understanding of the invention, reference should be made to the following detailed description, taken in connection with the accompanying drawings, in which:

FIG. 1 is a perspective view of an embodiment of the SiC prosthetic device in which SiC waveguides mounted onto the shank are used to direct the light from the optical emitters positioned on the support structure to the 3C SiC emitter lenses located at the tip of the 3C SiC shank.

FIG. 2 is a perspective view of an embodiment of the SiC prosthetic device in which a plurality of light emitters are fabricated directly on and extend longitudinally down the 3C SiC shank of the device through the use of a plurality of conductive traces. In this embodiment, 3C on 6H epitaxially grown light emitting diodes (LEDs) are formed directly on the shank thus eliminating the need for waveguides.

FIG. 3 is an image depicting one method for incorporating GaN. The image enumerates the processing steps required to generate a 3C-SiC/AlN/GaN stack using this method.

FIG. 4 is an image depicting a second method for incorporating GaN. The image enumerates the processing steps required to generate a 3C-SiC/6H-SiC/AlN/GaN stack using this method.

FIG. 5A-D are figures representing various diode structures. 5A) partially fabricated 3C PN diode; 5B) partially fabricated 6H PN diode; 5C) oxide passivated 3C PN diode; and 5D) oxide passivated 6H PN diode. (Philip G. Neudeck, David J. Larkin, Jonathan E. Starr, J. Anthony Powell, Carl S. Salupo, and Lawrence G. Matus, “Electrical Properties of Epitaxial 3C- and 6H-Sic p-n Junction Diodes Produced Side-by-Side on 6H-SiC Substrates”, IEEE Transactions On Electron Devices, Vol. 41, No. 5, May 1994, showing a dual color SiC LED structure). Using only 3C- and 6H-SiC highly biocompatible LEDs can be realized.

### DETAILED DESCRIPTION OF THE PREFERRED EMBODIMENT

In the following detailed description of preferred embodiments, reference is made to the accompanying drawings, which form a part hereof, and within which are shown by way of illustration specific embodiments by which the invention may be practiced. It is to be understood that other embodiments may be utilized and structural changes may be made without departing from the scope of the invention.

As used herein, the term “dura” refers to the fibrous covering over the brain and inside the skull.

As used herein, the term “shank” refers to the long thin SiC structure on the device that pierces the dura and makes intimate contact with the neural tissue.

As used herein, the term “support” refers to the top region of the prosthetic that remains outside the dura and contains the majority of the prosthetic's electrical subsystems.

As used herein, the term “ASIC” refers to an Application Specific Integrated Circuit. ASIC is the general purpose processer used on the device. The ASIC can handle some or all of the functions of the circuitry on the device and can work in conjunction with other circuitry on the device. For simplicity, all of these circuits collectively will be referred to as the “Interface Electronics”.

As used herein, the term “light emitter” refers to either a microfabricated LED or optical facet emitter on an optical waveguide onboard the device.

The design for optical neural probe 10 comprises two main structural components. Support structure 20 serves as the central communications, processing and power hub as well as a mechanical platform connecting shank 30 to prosthetic 10. ASIC subsystem 40 is located on support 20 and is capable of handling the communication and drive of optical devices onboard prosthetic 10. ASIC 40, typically fabricated from a silicon substrate with standard CMOS processing, is the general purpose processor for the prosthetic and handles all executive processes such as: (a) stimulation control with temporal and spatial precision through emitter selection; (b) communication control of collected electrical impulses and received commands pertaining to, but not limited to, optical stimulation; and (c) amplification, filtering, and otherwise processing of received electrical signals.

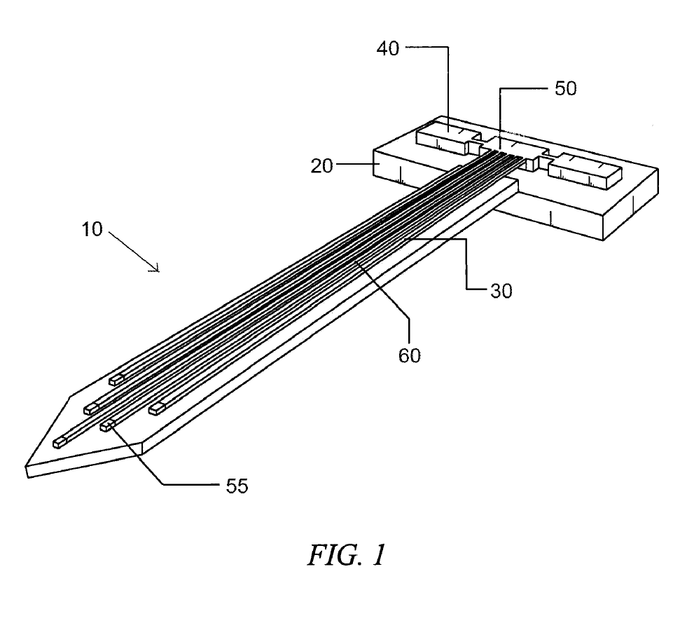

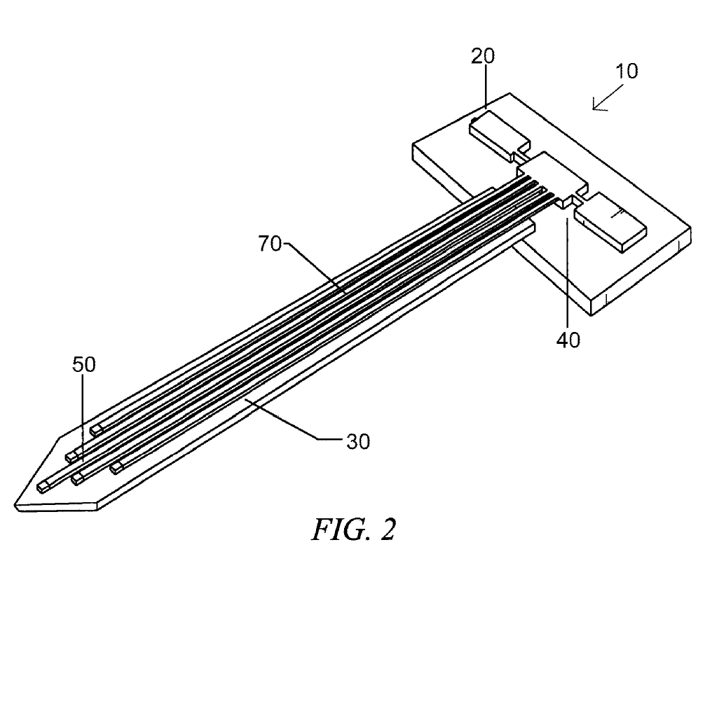

In use, the long, thin, silicon carbide shank structure 30 attached to support 20 makes direct contact with the targeted neural tissue (FIG. 1). Shank 30 contains optical facets and lenses capable of direct interface between prosthetic light emitters 50 and the surrounding neurons. Once implanted, device 10 interacts with the neural cells by providing wavelength specific light to the membrane of the targeted neural cells.

The generated light emerging from shank 30 (in either architecture) interacts with the genetically introduced ion channels (i.e. directly with the membrane ion channels), whereas many other devices (e.g. DBS devices) are more indirect and evoke electrical changes across large regions of space. Due to the size and density of the optical emitters on the present invention it is possible to activate single neurons (FIG. 1). In addition, this high density will make treating disorders such as epilepsy much more possible with the ability to find and treat small foci in the brain with long-term optical stimulation. This device increases longevity and reliability of the current standard due to the dramatic increase in biocompatibility that 3C-SiC provides over other similar biomaterials.

With little to no glial scar formation, the presented device maintains its light-to neuron interface long-term far exceeding the lifetime of similar designs. There are two variations of the shank emitter system. Each uses 3C-SiC as the outer most layer of the device and gallium nitride (GaN) as an inner buried layer. The buried GaN provides the active layer to realize a direct band gap blue LED which is needed for improved efficiency, whereas 3C-SiC allows direct interaction with the body environment as well as providing the material for the generation of a green/yellow LED.

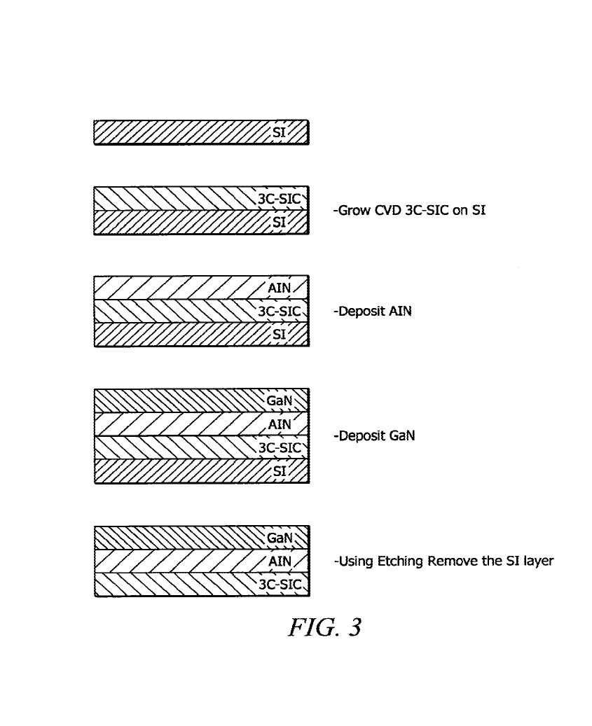

The first variation, shown in FIG. 3, begins with 3C-SiC grown on a Si substrate using CVD. GaN is then grown on 3C-SiC using vapor phase deposition by way of a thin (˜200 nm) aluminum nitride (AlN) buffer layer. After GaN growth the Si substrate is removed, leaving a GaN/AlN/3C-SiC layer stack. PN junctions will be formed to realize the LED's in both the GaN and 3C-SiC through either epitaxial growth of doped layers or through post-growth ion implantation/thermal annealing. Handle wafers will be used to allow processing of each side of the GaN/AlN/3C-SiC stack. Epitaxial growth and annealing temperatures must be below 1200° C. to avoid thermal damage to the GaN layer. When the devices are finished, they are bonded to the optical waveguide emitter structure using processes described in this document. The final stage ensures that the exposed AlN/GaN is hermetically sealed as these materials do not have an established biocompatibility level commensurate with neural implantation. The hermetic seal layer can be composed of amorphous SiC, diamond, or another biocompatible ceramic.

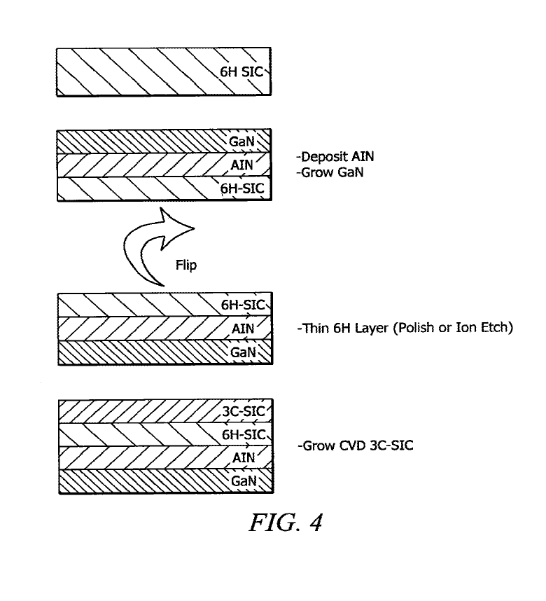

The second variation, illustrated in FIG. 4, generates 6H-SiC/AlN/GaN as a substrate for the growth of 3C-SiC. Deep reactive ion etching (DRIE) is used to etch the 6H-SiC substrate to a thin layer (˜1 um to 500 nm). The 6H-SiC surface is then mechanically polished flat and hydrogen etched to provide the surface for the growth of 3C-SiC, leaving a 3C-SiC/6H-SiC/AlN/GaN material layer stack. Again, each side of the stack (the 3C-SiC and GaN) is processed to generate PN junctions through epitaxial growth and ion implantation/thermal annealing as was stated in the previous section. The exact methods of attaching the stacks are examined in the next section. As with the previous section, any exposed GaN must be hermetically sealed from the body environment.

In one embodiment, support 20, normally fabricated from silicon with standard CMOS techniques, is connected to shank 30 through a high temperature anodic bonding process. A small region of the Si substrate is oxidized as well as the backside of the SiC shank assembly. The two oxidized surfaces are pressed together under a high voltage potential in excess of 8 kV applied by external plate electrodes and temperatures above 350° C. while clamped together. The process joins the two pieces tightly together. Shank 30 includes optical waveguide 60 or integrated SiC light emitters 50 and conductors 70 that deliver the light to the neural tissue. Shank 30 is implanted within neural tissue with support 20 residing just above the dura. Shank may also support microelectrodes and conductors 70, i.e. conductive traces, for electrical feedback from surrounding neurons.

In the same embodiment, support 20 is held to shank 30 using a biocompatible adhesive such as methyl 2-cyanoacrylate or ethyl-2-cyanoacrylate (Cyanoacrylate). Due to this union existing outside the dura, neurons would not come into contact with the adhesive preserving the neuron interface. Cyanoacrylate is a proven biocompatible adhesive and is often used in modern surgery.

SiC Optical Waveguide Shank Architecture

It has been previously established that SiC is optically transparent across a wide range of light wavelengths. This property is utilized in the architecture of the emitter design of the present invention to create a waveguide structure. Light is generated from emitters 50 located on support 20 (by LEDs or laser emitters) and is directed down the length of shank 30 and out into the environment using several SiC waveguides 60 and lens assemblies that are formed through lithography and dry etching on the surface of shank 30. Near the end of each waveguide 60 is a small lens structure 55 that guides the light directly to the neuron to be activated (FIG. 1).

Described herein is an example of how biocompatible waveguide structures can be made from layers of SiC. First, construction of waveguide 60 starts with a 6H-SiC or 4H-SiC substrate which is first patterned with photoresist to serve as a mask for an ion etching process. Then, using ion etching, rib waveguides are made by removing substrate material to the desired depth. Then, 3C SiC is grown over the top of the 6H sample using a hotwall CVD process conducted at/near 1300° C. in the presence of flowing gas precursors containing Si (commonly SiH4) and Carbon (commonly C3H8) (See P. G. Neudeck, A. J. Trunek, D. J. Spry, J. A. Powell, H. Du, M. Skowronski, X. R. Huang, M. Dudley “CVD Growth of 3C-SiC on 4H/6H Mesas Chemical Vapor Deposition” Special Issue: Silicon Carbide CVD for Electronic Device Applications, Volume 12, Issue 8-9, pp. 531-540, September, 2006).

The next step is using another lithography patterning step whereby waveguide 60 is etched to include conventional and/or Fresnel lens assemblies as well as other optical facets for light emission.

This architecture includes embodiments for feedback electrodes previously mentioned.

Integrated Emitter Architecture

In another embodiment of the device, biocompatible LED emitters 50 are directly fabricated on shank 30 through the use of SiC polytype selection and fabrication techniques. This configuration eliminates the need to guide light down the length of the prosthetic using waveguides but requires conductive traces 70 to power each light emitting diode (LED) structure to run along the length of the device.

SiC is a wide-bandgap semiconductor with bandgap ranging from 2.17 eV to 3.26 eV depending on polytype and doping concentration. Using a P-N junction arrangement light emitting diodes that range in wavelength 570 nm (yellow) to 380 nm (ultraviolet) can be created using 3C-, 6H-, and 4H-SiC polytypes. This is ideal as the most commonly used ion channel proteins are channelrhodopsin-2 and the halorhodopsin ion pump structures which are manipulated through wavelengths within this range. A wide range of colors on a single shank can be made of SiC, thereby facilitating a multitude of options in optical stimulation techniques. This multi-color, multi-channel, multi-mode capability allows for excitation and inhibition of multiple neuron types in-vivo.

LED emitters 50 can be constructed using layered SiC biomaterials. Having this aspect of the neural prosthesis made from SiC polytypes resulted in a wholly biocompatible prosthetic. Even if the device suffered catastrophic mechanical failure the material exposure will not be detrimental to the subject.

Described herein is an example of how biocompatible LEDs 50 can be made from layers of SiC. Construction of the emitter starts with a 4H- or 6H-(p type) SiC substrate. Next, p and n regions are ion implanted to form junctions in the substrate. Commonly, ion implantation of SiC uses aluminum and boron ions for “p” regions while nitrogen and phosphorus ions are used as “n” dopants. Then, the substrate is annealed at 1200° C.-1700° C. to activate the dopants and repair the damage done to the material by ion implantation. Next, 3C-SiC (n-type) epitaxial film is grown over the top of the substrate using a hotwall CVD process conducted at/near 1300° C. in the presence of flowing gas precursors containing Si (commonly SiH4) and Carbon (commonly C3H8). (See P. G. Neudeckl, A. J. Trunek, D. J. Spry, J. A. Powell, H. Du, M. Skowronski, X. R. Huang, M. Dudley “CVD Growth of 3C-SiC on 4H/6H Mesas Chemical Vapor Deposition” Special Issue: Silicon Carbide CVD for Electronic Device Applications, Volume 12, Issue 8-9, pp. 531-540, September, 2006)

Once the 3C epitaxial layer is formed it is also ion implanted with n and p dopants forming p-n junctions. Topside contacts are then applied to the 3C-SiC film directly over the ion implanted regions using either electron-beam evaporation or sputtering deposition. Next, these contacts are patterned using either lift-off or wet etch processes. Finally, these contacts are annealed at 600° C. for 20 min to form an ohmic contact that can be connected to conductive traces that carry current to the LED.

Another embodiment of the invention uses the base material stacks mentioned in the emitter section (3C-SiC/AlN/GaN or 3C-SiC/6H-SiC/AlN/GaN) as well as the LED generation detailed in the same section. The only processing difference is the final product would not be attached to the emitter or electronic section, but they would be standalone devices generated into a shank form using DRIE. To allow the GaN LED to interact effectively with the neural environment, a window is created in the 3C-SiC via DRIE processing. This window must leave a very thin (only 10 to 20 nm) layer of 3C-SiC so as to allow the blue light to pass through into the neural environment without significant loss. The previously mentioned bonding techniques are used to connect two shanks together at the GaN layers, generating an implantable planar device with 2 active sides. The remaining exposed layers of GaN/AlN or GaN/AlN/6H-SiC can be hermetically coated with amorphous SiC or diamond so as to maintain the required level of biocompatibility.

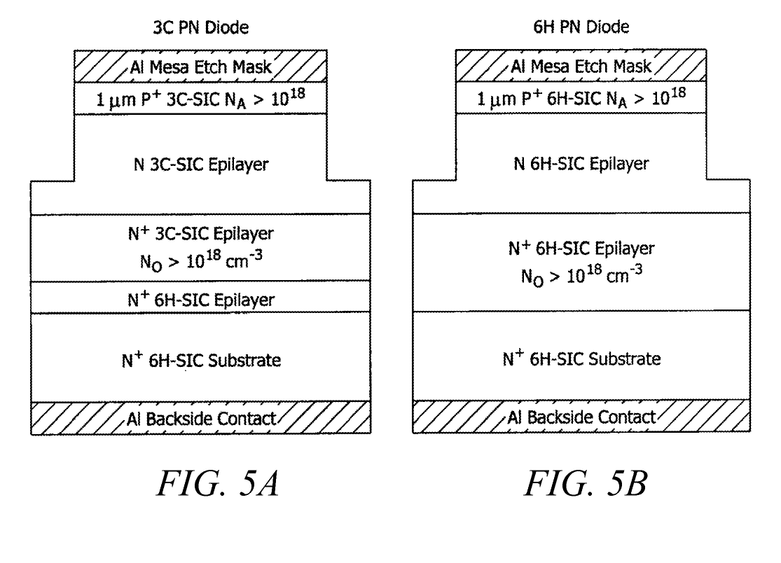

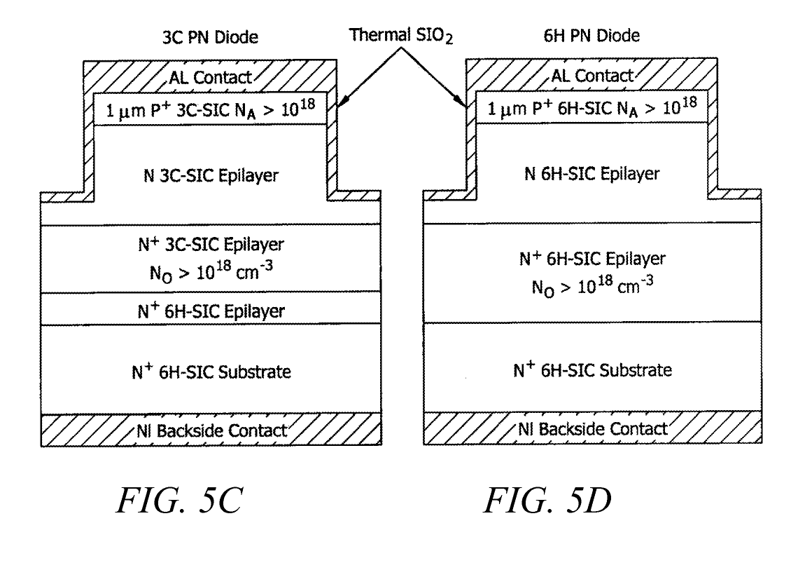

In another embodiment, LED emitters 50 are made of an alternating combination of 3C- and 6H-SiC materials. The junctions are layered so that multiple wavelengths can be realized from the same junction point. For biocompatibility reasons the 3C-SiC is grown on top of the 6H-SiC layer (FIG. 5).

In summary, the present invention offers a highly compact, biocompatible, and frequency agile light source with electrical feedback that is well suited for a number of in vivo optogenetics applications. The materials and methods used for its construction can be optimized as new ion channels and light stimulation techniques are discovered. The primary material of its construction, single crystal cubic silicon carbide (also known as β-SiC or 3C-SiC), is a highly biocompatible material that elicits a very low immune response and can be used in many ways to form all of the structures required to perform optical neural stimulation and feedback (light emitters, conductors, structural supports, and waveguides). Other such optogenetics-based prosthetics have not yet fully utilized this material for optogenetics.

It will thus be seen that the objects set forth above, and those made apparent from the foregoing disclosure, are efficiently attained. Since certain changes may be made in the above construction without departing from the scope of the invention, it is intended that all matters contained in the foregoing disclosure or shown in the accompanying drawings shall be interpreted as illustrative and not in a limiting sense.

It is also to be understood that the following claims are intended to cover all of the generic and specific features of the invention herein disclosed, and all statements of the scope of the invention that, as a matter of language, might be said to fall therebetween.

What is claimed is:

1. An implantable neuronal prosthetic device for optical neuron stimulation, comprising:
a support structure fabricated from silicon;
an electronics system disposed on the support structure providing a connection between the prosthetic device and outside world;
at least one shank mounted on the support structure, wherein the at least one shank is comprised of an outermost layer of single crystal cubic silicon carbide (3C-SiC); and
an optical system disposed on the at least one shank comprised of
at least one light emitter; and
at least one lens;
wherein the optical system generates a direct interface between the prosthetic device and surrounding tissue neurons.

2. The device of claim 1, further comprising a plurality of microelectrodes integrated into the at least one shank to provide feedback from targeted neurons.

3. The device of claim 2, further comprising a conductive trace connected to each of the plurality of microelectrodes.

4. The device of claim 3, wherein the conductive trace is comprised of a material selected from the group consisting of gold, platinum, platinum-iridium alloys, iridium oxide, stainless steel, tungsten, titanium nitride, graphene and heavily doped silicon carbide.

5. The device of claim 1, wherein the at least one light emitter is selected from the group consisting of light-emitting diodes and laser emitters.

6. The device of claim 1, wherein the electronics system is comprised of circuitry for wired communication.

7. The device of claim 1, wherein the electronics system is comprised of circuitry for wireless communication.

8. The device of claim 1, wherein the single crystal cubic silicon carbide layer is formed on a substrate selected from the group consisting of silicon, silicon carbide, single crystal diamond and gallium nitride.

9. The device of claim 1, wherein the optical system is further comprised of at least one optical light emitting P-N junction.

10. The device of claim 9, wherein the at least one optical light emitting P-N junction is comprised of at least one epitaxial layer of silicon carbide.

11. The device of claim 10, wherein the at least one epitaxial layer is a single layer of 3C-SiC.

12. The device of claim 10, wherein the at least one epitaxial layer is comprised of a layer of 3C-SiC grown directly on top of a layer of 6H-SiC.

13. The device of claim 1, wherein the optical system is further comprised of at least one waveguide structure.

14. The device of claim 13, wherein the at least one waveguide structure is fabricated from silicon carbide.

15. The device of claim 1, wherein the single crystal cubic silicon carbide is selected from the group consisting of 3C-SiC, 4H-SiC and 6H-SiC.

16. The device of claim 1, further comprising the at least one shank being a plurality of shanks arranged into a two- or three-dimensional matrix.

17. A method of manufacturing an implantable neuronal prosthetic device for placement in a patient for optical neuron stimulation, comprising the steps of:
providing a support structure;
depositing a substrate layer of a material selected from the group consisting of silicon, silicon carbide, single crystal diamond and gallium nitride on the support structure;
providing an electronics system on the support structure wherein the electronics system generates light and provides a connection between the prosthetic device and outside world;
mounting at least one shank on the support structure wherein the at least one shank is comprised of an outermost layer of single crystal cubic silicon carbide (3C-SiC); and
providing an optical system on the at least one shank wherein the optical system is comprised of
at least one light emitter; and
at least one lens;
wherein the optical system generates a direct interface between the prosthetic device and surrounding tissue neurons.

18. The method of claim 17, wherein the substrate layer is deposited on the support structure by a method selected from the group consisting of heteroepitaxial growth or chemical vapor deposition.

19. The method of claim 17, wherein the optical system is disposed on the at least one shank by forming at least one silicon carbide waveguide and at least one lens using dry etching and photolithography techniques.

20. The method of claim 17, wherein the optical system is disposed on the at least one shank by forming at least one optical light emitting P-N junction.

21. The method of claim 17, further comprising attaching at least one electrode to the at least one shank to receive feedback from surrounding neurons.

22. The method of claim 17, further comprising arranging a plurality of the shanks into a matrix.
